(19) United States
(12) Patent Application Publication
(10) Pub. No.: US 2003/0228239 A1
(43) Pub. Date: Dec. 11, 2003

(54) Method of inactivating prions

(75) Inventors: Richard S. Meyer, Tacoma, WA (US); Paul Brown, Bethesda, MD (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Prion Inactivation Partners

(21) Appl. No.: 10/167,115

(22) Filed: Jun. 10, 2002

(51) Int. Cl. A61L 2/00

(52) U.S. Cl. 422/39

(57) ABSTRACT

A method for inactivating prions comprises subjecting a substrate containing prions to one or more pulses of ultra high pressures for a predetermined period of time. The pressures is preferably at least 480 MPa for at least one second. The substrate and the prions are adiabatically heated to elevated temperatures at which the prions are inactivated.

### FIELD OF THE INVENTION

This invention relates to a method for inactivating prions, and more particularly to a method of inactivating prions by subjecting the substrate in which the prions are present to ultra high pressures, and in a preferred embodiment, to a method of inactivating prions in a food product by subjecting the food product to ultra high pressure.

### BACKGROUND OF THE INVENTION

Prions are aberrant proteins that are present in the brains of animals. Aberrant prions when present cause normal proteins to reconfigure. This modification of normal proteins in turn cause an amyloid deposit (plaque) to form in the brain. Normal enzymatic processes do not remove the plaque. The result is a debilitating neural disease that in its several forms affects both animals and humans. Natural infections have so far been restricted to sheep and goats (scrapie), and to deer and elk (chronic wasting disease). Sheep have transmitted scrapie in ‘unnatural infections’ to mink (transmissible mink encephalopathy) and cattle (‘mad cow disease’) via contaminated carcass feed, and the same vehicle transmitted disease to a variety of exotic zoo ungulates, domestic and zoo felines, and zoo primates. Carcasses from which as much meat as possible has been manually removed, but which, historically, still included heads and vertebral columns, are subjected to a process of compression to yield a paste of ‘mechanically recovered meat’ that was permitted to be added to a variety of pre-cooked meat products such as hot dogs, sausages, meat patties, luncheon meats, beef stews, pureed baby food meats, etc. It is now abundantly clear that central nervous system tissues were entering the human food chain through this unadvertised vehicle, and that they were the most likely cause of human infection.

Prions are very difficult to inactivate. The World Health Organization currently recommends subjecting prions to a temperature of 134° C. in a 1 N sodium hydroxide solution to deactivate them. While this process is effective, it is not practical for inactivating prions in food products, medical products, blood and blood supplies, animals, and pharmaceutical extracts.

### SUMMARY OF THE INVENTION

The present invention therefore provides a method of inactivating prions and particularly prions in a substrate such as foods, medical products, blood and blood supplies, animal feed, pet food, vaccines, cell culture nutrients, and pharmaceutical extracts and products, by subjecting the substrate to an ultra high pressure for a predetermined period of time to heat the substrate and the associated prions adiabatically from an initial temperature to a final temperature up to 400° C. In a preferred form of the invention, the substrate is subjected to multiple pulses of ultra high pressure, each for a predetermined period of time. The ultra-high pressure pulses effectively inactivate the prions to a level at which they no longer present a hazard from the ingestion or use of the substrate by humans.

### DETAILED DESCRIPTION OF THE PREFERRED EMBODIMENT

The present invention is applicable to a broad variety of substrates in which prions may exist. The substrates include, but are not limited to, foods, medical products, blood, blood supplies, animal feed, pet food, vaccines, cell culture nutrients, and pharmaceutical extracts and products. The present invention is applicable to prions in any substrate, however, the invention works best with a relatively incompressible substrate. The prions are inactivated by subjecting them to an ultra high pressure for a predetermined period of time.

As used herein, the term “prion inactivation” means partial or total destruction of the replicating property of prions (infectivity). Also note that tests for the prion protein (PrPres), which can be performed much more quickly than bioassays, are a reliable index of infectivity. The ultra high pressure to which the substrate is subjected is in the form of a very short pulse, a very long pulse, or multiples thereof. If multiple pressure pulses are employed, it is preferred that the pressure on the substrate be reduced to at or near atmospheric pressure between each of the pulses. The application of pressure instantaneously and uniformly raises the food temperature to a final temperature desired for inactivation of the prions. The substrate is raised to its final temperature by a combination of its initial temperature and the adiabatic heating caused by the increase in pressure. It is preferred, of course, that the increase in pressure occur rapidly, however, it can be applied over time, if desired. It is also preferred that the vessel in which the substrate is being heated be adequately insulated so that little heat loss occurs. This will result in the maximum rise in temperature resulting from adiabatic heating.

It is preferred that the final temperature range up to 400° C., more preferably from 40° C. to 200° C., and most preferably from 40° C. to 145° C. The pressures employed can also vary. The ultra high pressure applied to the substrate is preferably at least 480 MPa, and preferably between 480 MPa and 970 MPa, and more preferably, to a pressure of from 480 MPa to 830 MPa.

The initial temperature for the substrate may be room temperature, on the order of 25° C. The substrate can be preheated to an initial temperature higher than room temperature, for example, on the order of 70° C. or higher. Of course, preheating the substrate to a temperature above room temperature will increase the final temperature (resulting from a combination of the preheating and the adiabatic heating) that will be higher than if the substrate is subjected to pressure beginning only at room temperature.

The instant adiabatic heating and cooling that occurs upon pressurization and depressurization minimizes exposure of the substrate to heat (prolonged exposure to heat causes damage to the flavor, texture, color, and other characteristics of a food product, for example), the subjection to ultra high pressure sufficient to achieve prion inactivation when carried out as set forth above. The substrate may be preheated, for example, in a water bath, before being placed in a pressure vessel. It can also be preheated in a commercial plate heat exchanger or surface heat exchanger, or can be heated in the pressure vessel equipped with its own heater. Pressurization equipment having both heating and cooling capacity in the pressure vessel is available, for example, from ABB Autoclave Systems, from Flow Industries, and from Engineered Pressure Systems.

During each pressurization cycle, the principle of adiabatic heating (and cooling with the release of pressure) results in an increase (or decrease) of about 30-45° C. in the temperature of the substrate (or higher with pressures beyond 830 MPa). The actual increment is the function of both the initial temperature and the amount of pressure applied. For example, if 400 MPa is used to pressurize a substrate preheated to 99° C., the adiabatic heat increase is about 25° C., but if 690 MPa is applied, the increase in temperature is about 32° C. Thus, applying ultra high pressure to a preheated substrate results in a deactivation temperature that is much higher than the initial temperature of the substrate. By taking the adiabatic temperature rise into account, the elevated temperature exposure of the substrate can be minimized and controlled within a second. Thus, the damage caused by prolonged and excessive exposure to elevated sterilization temperatures can be avoided and the flavor, texture, and color, or other desired characteristic of the substrate, can be protected with minimal or no change. The preheat temperature and pressure should be selected in accordance with the present invention to reach the time and temperature combination that inactivates the prion in the substrate with a minimum exposure to high pressure and temperature.

Pressurization for purposes of the methods disclosed herein may be achieved using any commercially available device capable of delivering the requisite high pressures and high temperatures. Prior to pressurization, the food products usually are sealed inside a suitable container, such as a plastic bag, can, or other container, or may be pumped through a heat exchanger and then into a pressure vessel in bulk, and packaged aseptically in sterile containers after the pressurization step.

Prior to application of high pressure, it is preferred that air be removed from the container in which the substrate is placed and from the pressurized vessel. If air is present during the processing, compounds that contribute to flavor in the food, for example, might become oxidized. Moreover, because air compresses at high pressures, its presence in the vessel would result in a loss of efficiency, and thus a lower adiabatic temperature rise. Also, the air could react with plastic packaging material and cause scorching or burning.

### EXAMPLES

The following Examples set forth herein are intended to be illustrative of how to make and use the invention. They are not intended in any way to delimit its scope or otherwise detract from its applicability to a broad variety of substrates in which prions are present.

Pressure/temperature tests. Each of the samples was packed for testing as follows: Pretreated samples were initially packed and vacuum sealed using an impulse sealer in 5 cm by 8 cm laminated plastic foil pouches (0.75 mm nylon+2.25 mm polyethylene) then repacked and vacuum sealed again with an impulse sealer in 5 cm by 8 cm metallized pouches (12 micron polyester-outer layer, 15 micron nylon, 12.5 micron aluminum foil, 102 micron polypropylene-inner layer). Two pouches were then placed in a 200 ml high density polyethylene bottle (4.2 cm diameter and 9.3 cm long) and 95° C. castor oil was added to the bottle to displace all the air and the bottle was sealed with the bottle cap. The bottle and contents were placed in a 95° C. water bath until ready for placement in the metal cylinder with the diaphragm. The bottles and contents were held for minimum of 15 minutes in the 95° C. water bath to equilibrate the temperature.

The pressure vessel has a pressure chamber size of 0.7 liters. The vessel pressurizes in 20 to 30 seconds, depending on end pressure and depressurizes in 10 to 20 seconds. The vessel has an external heater which was set to 95° C. The vessel was operated to 690 MPa, 1000 MPa, and 1200 MPa pressure. Castor oil is used to fill the vessel chamber volume remaining after the sample container is inserted. The castor oil was preheated to 95° C. and added prior to every run. (The castor oil in the chamber is removed after every run because it cools below 95° C.) The bottle containing the product sample was placed in a metal cylinder, which was in turn placed in the pressure chamber. The metal cylinder (8 cm diameter and 18 cm long) is made of stainless steel and has an inner woven fabric diaphragm with a screw on top cap and a solid cap base. The sidewalls of the cylinder have large openings to allow the fluid pressure to act upon the diaphragm contents. The cylinder is sized to easily fit inside the vessel chamber. The cylinder cap has attachments for plugging in a thermal couple lead wire. The product sample container was placed in a high density polyethylene bottle with a screw on cap, which was in turn placed in the diaphragm. Hot water (95° C.) is added to the diaphragm after the bottle is inserted to displace all air. The cylinder cap is then screwed on and the cylinder is then placed in the vessel chamber to which hot (95° C.) castor oil has been previously added. The vessel chamber is capped, the thermal couple leading into the diaphragm chamber is connected and the vessel yoke is moved over the cap to hold pressure. The vessel pressure and temperature are measured and recorded on a computer every 2 seconds. For the tests recorded below, the pressure pulses were applied for 60 seconds, with a 30 second ambient pressure pause between pulses.

The prion containing material was derived from whole hamster brains taken from animals sacrificed at a terminal stage of illness following intracerebral inoculation with hamster-adapted sheep scrapie strain 263K, and stored at −70° C. until used. This strain of scrapie is widely used for the exceptional resistance of its prions to physical and chemical inactivation procedures and high infectivity titer (about 10¹⁰ LD₅₀ per gram of brain). Whole hamster brain was homogenized and was tested by itself as a control. It was also injected into a blood plasma and tested. In addition, whole hamster brains were thawed and homogenized with a commercial meat product (hot dogs) using a sealed top container in a mechanical homogenizer at a ratio of 1 part brain to 99 parts meat (1% by weight), which yielded a pretest infectivity level of approximately 10⁸ LD₅₀ per gram of meat. Samples were distributed in separate watertight plastic packets and subjected to various pressures using three or ten pulses for a period of one minute. One sample was subjected to a single pulse for five minutes.

A plasma sample was also prepared in a manner similar to the meat samples. Separated fresh frozen plasma was utilized. The fresh plasma was mixed with a clarified 10% scrapie hamster brain suspension at a ratio of 10 ml per 90 ml of plasma (1:10 dilution). The estimated infectivity input was about 10⁸ LD₅₀ per ml. A 3 ml aliquot was distributed into a test packet.

The materials and methods utilized to determine reduction in activity and to detect infectivity are set forth below.

Western Blots Test to Detect PrPres (“Prion Protein”): A 20 to 40 mg aliquot of thawed sample is added to a sufficient volume of Tris buffered saline to make a 10% w/v suspension. Tissue is disrupted by repetitive pipetting, 2 freeze/thaw cycles, and sonication. SDS-PAGE (sodium dodecylsulfate-polyacrylamide gel electrophoresis) is performed as follows: 100 μl of each sample is incubated with proteinase K and then mixed with an equal volume of 2× concentrated SDS sample buffer. Undiluted and serially diluted samples (in half-log steps) are titrated to determine the end-point dilution for PrPres detection. 15 μl of the SDS/sample mixtures are loaded onto 12% polyacrylamide gels, electrophoresed, and the charge/size-separated proteins are transferred onto nitrocellulose paper. PrPres is detected by a labeled anti-prion antibody.

Bioassays to Detect Infectivity: A 0.3 to 0.5 g aliquot of thawed sample is added to a sufficient volume of phosphate-buffered saline (PBS) to make a 10% w/v suspension, which is homogenized and then serially diluted in one-log steps in PBS. Samples are inoculated into groups of hamsters: 50 μl of each (undiluted or diluted) sample is inoculated into the right cerebral hemisphere of each animal. The animals are observed for clinical signs of scrapie during a period of one year following inoculation, and the brains of all dying animals are examined for the diagnostic presence of PrPres using a standard Western blot method. In the table below ND, means no prions detected.

Results of the tests are set forth in Table 1 below.

TABLE I
SUMMARY OF WESTERN BLOT PrP^{SC} AND
BIOASSAY INFECTIVITY CLEARANCE
(AS OF 30 May 2002)

| | | | | Bioassay |
|---|---|---|---|---|
| | | | Log₁₀ | Log₁₀ |
| MPa Start | Start Temp | Number of | PrP^{SC} | clearance |
| Pressure | (° C.) | 1 min pulses | reduction | (8 log input) |
| BRAIN | | | | |
| 1200 | 95 | 3 | 3 | 4 |
| HOT DOGS | | | | |
| 1200 | 95 | 10 | ≧4.5 | 4 |
| 1200 | 95 | 3 | ≧4.5 | ≧4 |
| 1200 | 95 | 1 (5 min) | 3.5 | ≧4 |
| 1000 | 95 | 10 | 4.5 | ND |
| 1000 | 95 | 3 | 3.5 | ND |
| 690 | 95 | 10 | 2 | 3 |
| 690 | 95 | 3 | 2 | 3 |

A good correlation was found between the Western blots and the infectivity clearance. The samples pressurized to 1200 MPa were totally inactivated, whether subjected to 3 to 10 sixty second pulses. Multiple pulses were equal to or better than a single pulse for 5 minutes. Results also show that samples pressurized to 1000 MPa were inactivated as much as 1200 MPa for 10 pulses but not as much using 3 pulses (blot results only). Finally, samples pressurized to 690 MPa were only partially inactivated.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for inactivating prions present in a substrate comprising:
subjecting said substrate containing prions to at least one pulse of ultra high pressure for a predetermined period of time to heat said substrate containing prions adiabatically from an initial temperature to a final temperature up to 400° C.

2. The method of claim 1, wherein the final temperature ranges up to 400° C.

3. The method of claim 2, wherein the final temperature ranges from 40° C. to 200° C.

4. The method of claim 3, wherein the final temperature ranges from 40° C. to 145° C.

5. The method of claim 1, wherein said ultra high pressure is at least 480 MPa.

6. The method of claim 5, wherein said pressure ranges from 480 MPa to 970 MPa.

7. The method of claim 6, wherein said pressure ranges from 480 MPa to 830 MPa.

8. The method of claim 1, wherein the initial temperature is room temperature.

9. The method of claim 1, wherein said initial temperature is 40° C.

10. The method of claim 1, wherein said substrate is subjected to a plurality of pulses of ultra high pressure.
